(19) United States
(12) Patent Application Publication
(10) Pub. No.: US 2007/0129762 A1
(43) Pub. Date: Jun. 7, 2007

(54) Cardiac pacemaker with dynamic conduction time monitoring

(76) Inventor: Seth Worley, Lancaster, PA (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 11/292,745

(22) Filed: Dec. 1, 2005

(51) Int. Cl. A61N 1/00

(52) U.S. Cl. 607/9

(57) ABSTRACT

In accordance with the present invention, a cardiac device is provided comprising a first electrode for sensing an electrical pulse in an atrium, a second electrode for sensing an electrical pulse in an atrium, a microcontroller coupled to the first electrode and the second electrode for determining time between the first and second pulses, and an electrode for stimulating a chamber of a heart wherein the stimulation is based on the first and second pulses.

### BACKGROUND OF THE INVENTION

This invention relates to methods of controlling the pacing of a heart.

In the normal human heart, the sinus node, generally located near the junction of the superior vena cava and the right atrium, constitutes the primary natural pacemaker initiating rhythmic electrical excitation of the heart chambers. The cardiac impulse arising from the sinus node is transmitted to the two atrial chambers, causing a depolarization known as a P-wave and the resulting atrial chamber contractions. The excitation pulse is further transmitted to and through the ventricles via the atrioventricular (A-V) node and a ventricular conduction system causing a depolarization known as an R-wave and the resulting ventricular chamber contractions.

Disruption of this natural pacemaking and conduction system as a result of aging or disease can be successfully treated by artificial cardiac pacing using cardiac pacing devices, including pacemakers and defibrillators, which deliver rhythmic electrical pulses or anti-arrhythmia therapies to the heart at a desired energy and rate. One or more heart chambers may be electrically paced depending on the location and severity of the conduction disorder. In addition, recent advances in pacing for ventricular dyschrony, referred to as cardiac resynchronization therapy, requires the ventricle(s) to be paced before normal conduction through the AV node depolarizes the ventricles.

Modern implantable pacemakers and defibrillators possess numerous operating parameters, such as pacing pulse energy, pacing rate, sensing threshold, pacing mode, etc., that must be programmed by the clinician to satisfy individual patient need. One specific parameter that must be programmed is the atrio-ventricular delay (AV delay), which is the time period between atrial electrical activity and electrical activity in the ventricles. It is known that the A-V delay may be optimized empirically at a fixed point in time, through the use of echocardiograms, or through invasive evaluation. In the literature, the optimum value of the AV delay has generally been defined as that delay value that produces the maximum stroke volume for a fixed heart rate or the maximum cardiac output for a sinus node driven heart rate.

In the case of cardiac resynchronization therapy, the optimal AV delay timing is obtained when the onset of left ventricle (“LV”) contraction occurs immediately upon completion of the left atrium (“LA”) contribution (also referred to as Left Atrial Kick) in late diastole. At this moment, the LV filling (preload) is maximum, and what is known in the art as the Frank Starling Relationship between LV stretch and LV contraction is the greatest. This will result in maximum LV stroke volume ejection, and thus realization of the maximum Cardiac Index/Cardiac Output.

With present day state-of-the-art, programmable, implantable pacemakers, a cardiologist is able to periodically program into the device an AV delay value that yields an optimum stroke volume. One way of accomplishing that technique is for example, by using external instrumentation such as a Doppler flow meter to measure changes in cardiac output as the AV delay interval for the pacer is systematically changed. Such an approach at optimization is not only time consuming, but may only be appropriate for the patient at the time that the testing and setting of the AV delay interval is made.

Moreover, in order to achieve full atrial contribution to LV filling, optimal programming of the AV delay in pacemakers must be long enough to achieve full depolarization of the pacing stimuli (sinus or paced). However, optimal programming of the AV delay must be short enough to insure optimal filling from atrial contraction. In an overly long AV delay situation, the filled left ventricle has more time to let blood flow back into the left atrium before contraction starts (mitral regurgitation), which reduces the cardiac output. Further, in the case of cardiac resynchronization therapy (herein referred to as “CRT therapy” or “CRT”), if the AV delay is too long, ventricular contraction will occur as a result of conduction through the AV node rather than pacing which reduces the cardiac output and increase mitral regurgitation independent of LA contraction. In an overly short AV delay situation, the atrium may contract at a time when the mitral valve is closing or closed, reducing filling and thus cardiac output. With the proper AV delay programmed into the pacemaker, the maximum cardiac output requirements (exact synchronized filling of LV, optimal LV filling period, and optimal preloading of LV) are met. In the case of CRT, this also means that the ventricles will be fully depolarized by the pacing leads and not via conduction through the AV node. To insure optimal AV filling in CRT, changing the AV delay alone may not be sufficient because of prolonged inter-atrial conduction times resulting in delay of left atrial activation beyond the intrinsic AV delay. In this case, pacing the LA and adjusting the AV delay for LA pacing will be required to insure optimal AV filling.

Echocardiography based optimization of the atrioventricular delay in patients has been used to insure that left atrium contraction occurs before closure of the mitral valve. Resting echocardiography based optimization of the atrioventricular delay is time consuming, expensive, operator dependent and may not be appropriate for the active patient. It is desirable to be able to precisely program the optimal AV delay for each patient without relying on empirical data or echocardiography. Further, the optimal AV delay varies acutely with the patient's level of activity, and varies chronically depending on conditions that cause hypertrophy, fibrosis or remodeling. Thus it is ideal that the AV delay be continuously adjusted based on the patient's acute and chronic physiology

### SUMMARY OF THE INVENTION

One object of the present invention is to provide a method of dynamically insuring that the AV delay is optimal for ventricular filling. Optimal AV filling is maintained by measuring the electrical activity at two or more sites in the atria and programming the atrioventricular delay of a cardiac device and/or pacing the left atrium and programming the AV delay. Such a method of dynamically optimizing AV filling either by changing the AV delay or pacing the LA and changing the AV delay would overcome the shortcomings of the prior art and allow the atrioventricular delay to be programmed without relying on empirical data or echocardiography based optimization. For example, if the AV delay required to achieve full atrial contribution to LV filling resulted in the ventricles being activated through native conduction, it would not result in the optimal AV delay. Only by pacing the LA and adjusting the AV delay would optimal filling for CRT be insured.

In accordance with the present invention, a cardiac device is provided comprising a first electrode for pacing or sensing an electrical pulse in an atrium, a second electrode for pacing or sensing an electrical pulse in an atrium, a microcontroller coupled to the first electrode and to the second electrode for determining time between the first and second pulses, and an electrode for stimulating one or more chambers of a heart wherein the stimulation is based on the first and second pulses. The sensed interval (“electrical pulse”) between the first sensed or paced pulse and the second sensed pulse is the inter-atrial conduction time when the leads are in different atria and intra-atrial conduction time if the two electrodes are in the same atrial chamber. In one embodiment, when single chamber electrodes are employed the first electrode and second electrode may be in the same atria or in different atria. In another embodiment, the first electrode is in a right atrium, the second electrode is in a left atrium whereby the third and/or forth electrode(s) are positioned in the ventricle(s) Further, when compound electrodes are employed the electrode for stimulating one or more ventricles may be incorporated within the first compound electrode or may be incorporated in the second compound electrode or both A compound electrode is designed to pace and/or sense two or more independent sites or heat chambers. In one embodiment, stimulation is provided only to one of the left or right ventricles. Depending on the sinus rate and the inter-atrial conduction time, any or all of the atrial and ventricular electrodes may be paced. Moreover, the invention may further include one or more coils or other means for shocking one or more chambers of the heart.

In accordance with the present invention, a cardiac device is also provided comprising a microcontroller, a first lead in communication with the microcontroller having at least a first electrode for pacing or sensing cardiac events in a first atrium, a second lead in communication with the microcontroller having at least a second electrode for pacing or sensing cardiac events in a second atrium, and at least one electrode for pacing or sensing cardiac events in one or more heart chambers, whereby the microcontroller measures a delay between paced or sensed cardiac events of the first electrode and the sensed event of the second electrode, and whereby the microcontroller directs the stimulation pulse based on the delay. With cardiac resynchronization therapy, the delay between paced or sensed cardiac events of the first electrode and the sensed event of the second electrode may exceed a given interval such that intrinsic AV node conduction can occur. In this instance, optimal AV filling also requires pacing the left atrium. The first and second atria may be the same atria or may be different atria. In one embodiment of the invention, the first electrode is in a right atrium and may be a tip electrode, a ring electrode, or both. In another embodiment of the invention, the second electrode is in a left atrium and is selected from a tip electrode or a ring electrode or both. The electrode for sensing or stimulating the ventricles may be a third electrode and may be connected to the second lead as part of a compound lead. Other electrodes known in the art may be substituted in keeping with the apparatus and methods of the present invention. The microprocessor may cancel the stimulation pulse to one or both of the ventricle(s) if one or more of the electrodes in the ventricles sense a cardiac event occurring in the one or both ventricles before the end of the AV delay determined by the inter-atrial measurement. The stimulation pulse to the ventricles may be to one or both ventricles. When stimulation of two ventricles occurs, they may be paced simultaneously or with a delay. Each ventricle may be stimulated at one or multiple locations. Moreover, the third single chamber electrode may terminate in a left ventricle and may be selected from a tip electrode or a ring electrode or both. In addition, the cardiac device may also include one or more coils so as to provide shocking therapy to one or more chambers of the heart.

In accordance with the present invention, a cardiac device is also provided comprising a microcontroller, a first lead connected to the microcontroller having at least a first electrode for pacing or sensing cardiac events in a right atrium, a second lead connected to the microcontroller having at least a second electrode for pacing or sensing cardiac events terminating in a left atrium, and at least one electrode for pacing or sensing cardiac events in a left ventricle, whereby the microcontroller measures a delay between cardiac events sensed or paced by the first and second electrodes, and whereby the microcontroller directs a stimulation based on the measured delay. It should be noted that the leads utilized as part of the current invention may be compound leads. A compound lead is a lead containing two or more individual leads, with each individual lead terminating in at least one electrode. The microprocessor may cancel the stimulation pulse to one or both of the ventricle(s) if one or more of the electrodes in the ventricles sense a cardiac event occurring in one or both ventricles before the end of the AV delay determined by the interatrial measurement. Further the cardiac device may include a third lead that contains electrodes for sensing, pulsing, and/or shocking. Indeed, the device may also contain one or more elements so as to provide shocking therapy to one or more chambers of the heart.

### BRIEF DESCRIPTION OF THE DRAWINGS

FIG. 1 is a diagram showing several cardiac electrical events and their timing in relation to one another.

FIG. 1A is a diagram showing a spontaneous cardiac electrical event recorded from the body surface, and two sites in the atria, most commonly the right atrium and left atrium, and their timing in relation to one another in a patient in sinus rhythm with normal inter-atrial conduction time and normal total atrial activation time.

FIG. 1B is a diagram showing a spontaneous cardiac electrical event recorded from the body surface, and two sites in the atria, most commonly the right atrium and left atrium, and their timing in relation to one another in a patient in sinus rhythm with prolonged inter-atrial conduction time and prolonged total atrial activation time.

FIG. 1C is a diagram showing a paced cardiac electrical event recorded from the body surface, and two sites in the atria, most commonly the right atrium and left atrium, and their timing in relation to one another in a patient whose atrium is being paced using a right atrial lead.

FIG. 2A is a functional flowchart illustrating the operation of an embodiment of the cardiac device.

FIG. 2B is an alternate functional flowchart illustrating the operation of a second embodiment of the cardiac device.

FIG. 3A is a basic block diagram of an implantable multi-chamber cardiac device of the present invention, shown in electrical communication with three leads and a plurality of electrodes in a heart.

FIG. 3B is a basic block diagram of an implantable multi-chamber cardiac device of the present invention, shown in electrical communication with four leads and a plurality of electrodes in a heart.

FIG. 4A is a simplified, partly cutaway view of an implantable multi-chamber cardiac device of the present invention, shown in electrical communication with three leads implanted into a patient's heart for delivering multi-chamber stimulation and/or shock therapy.

FIG. 4B is a simplified, partly cutaway view of an implantable multi-chamber cardiac device of the present invention, shown in electrical communication with three leads implanted into a patient's heart for delivering multi-chamber stimulation and/or shock therapy.

### DETAILED DESCRIPTION

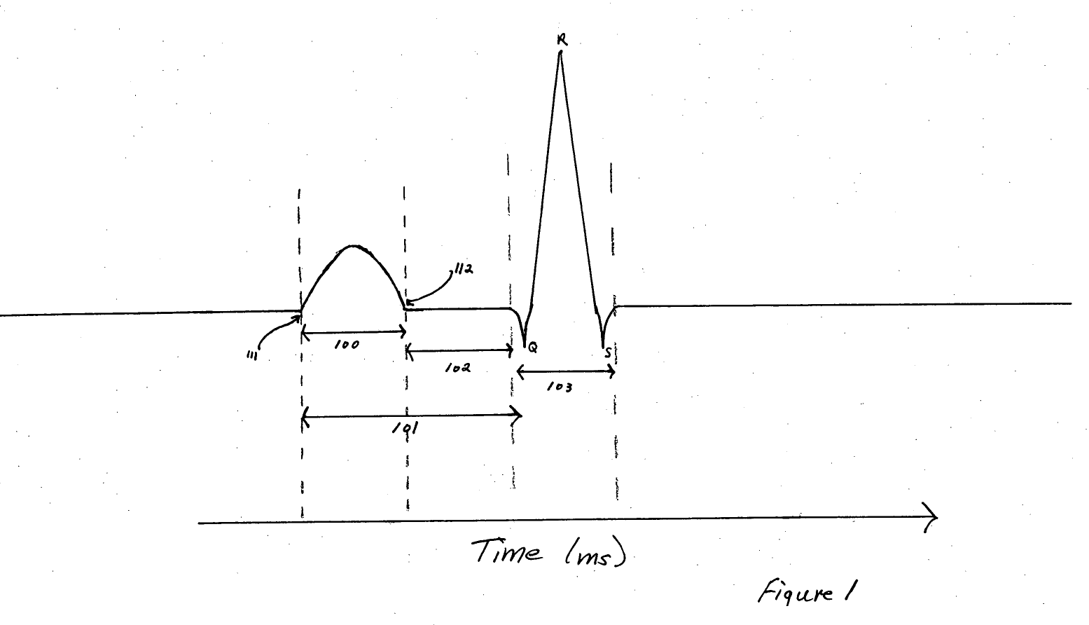

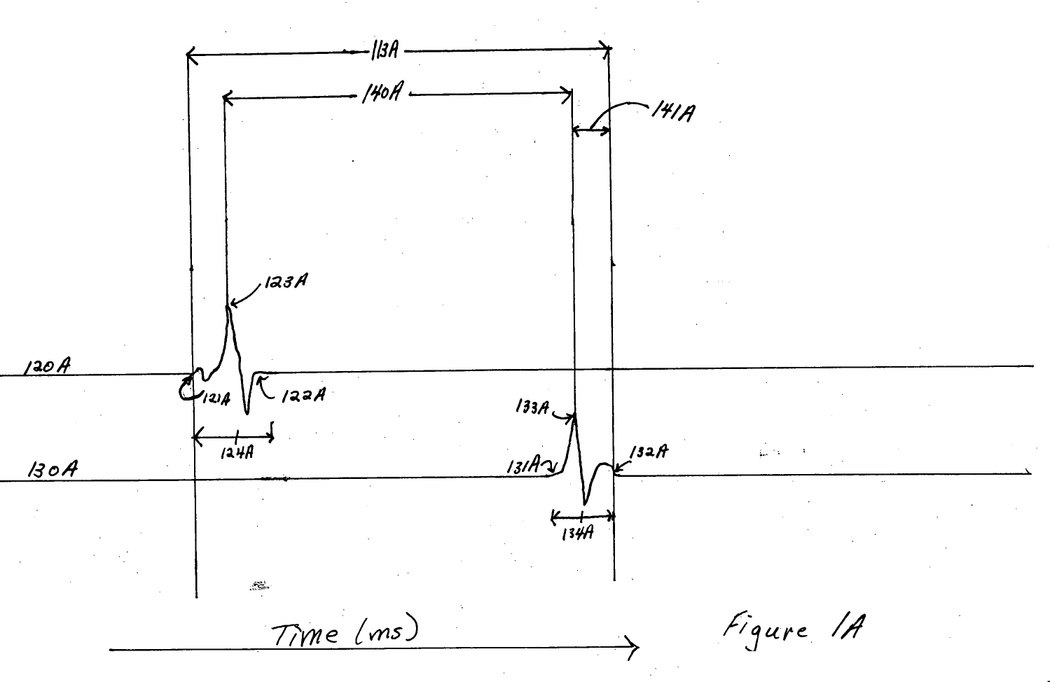

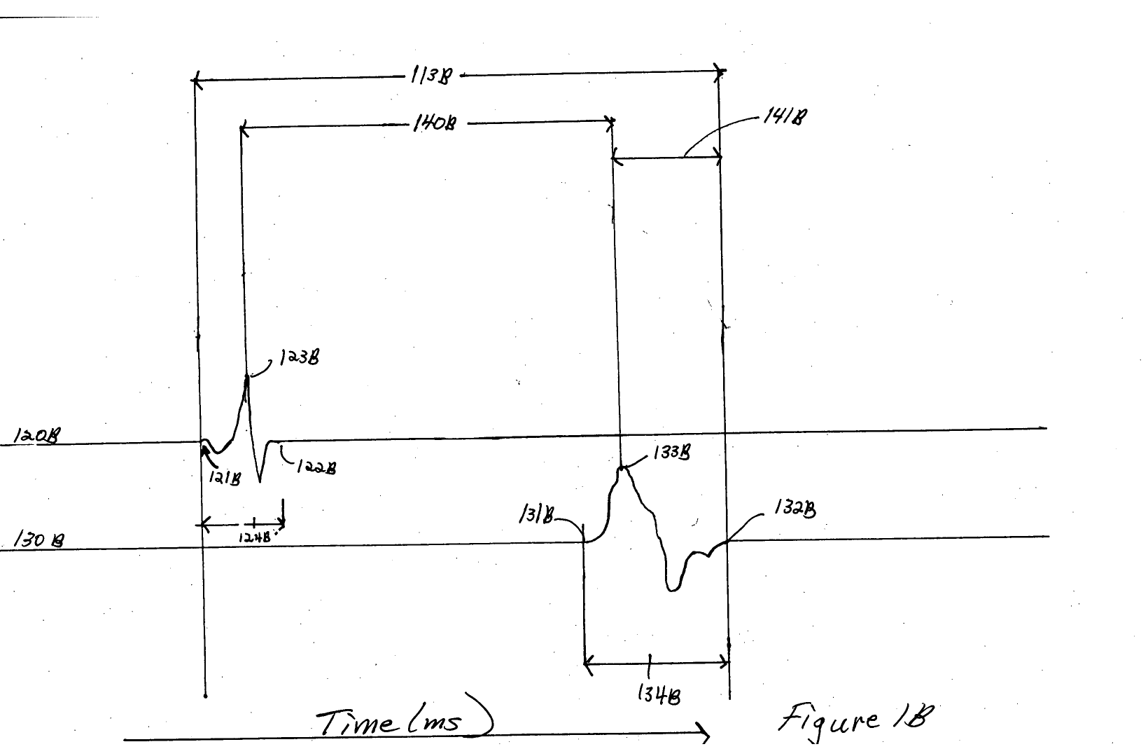

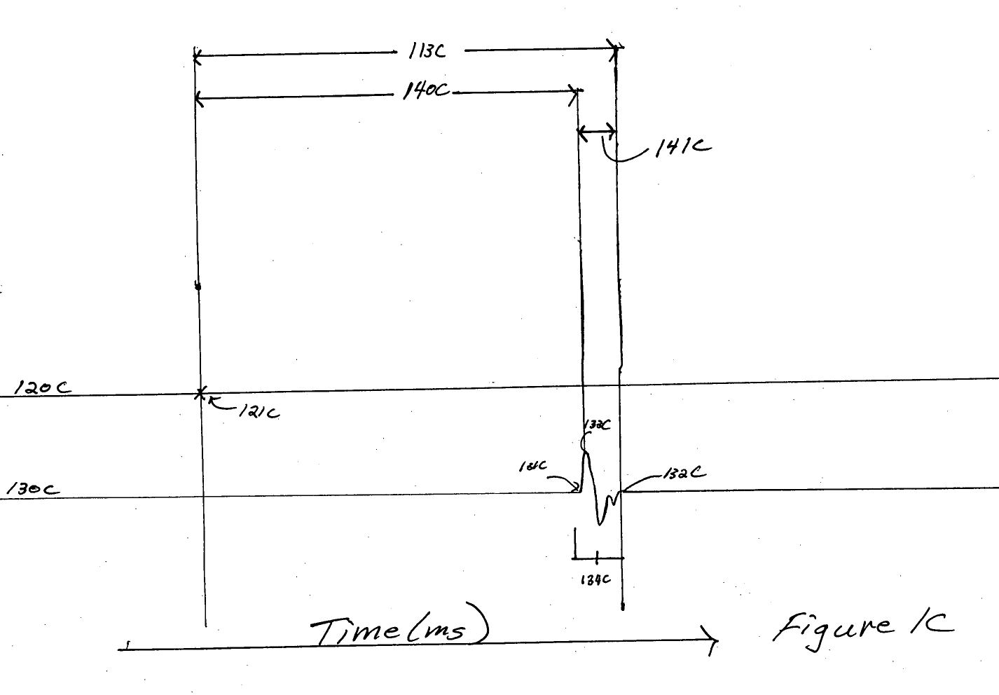

Referring to FIG. 1, which depicts the electrical activity of a heart as recorded by electrocardiography from the body surface, the P-wave 100 represents the wave of depolarization that spreads from the sino-atrial node through the atria on the body surface electrocardiogram (“ECG”). Said another way, the P-wave is the electrical activity generated by depolarization of the atrium as recorded on the body surface by the ECG. The duration of the P-wave in any one of the 12 leads of the ECG may vary according to position of the ECG lead relative to the electrical vector created by the depolarizing atrium. The P-wave usually ranges from 80 ms in duration to 100 ms in duration. The P-wave is generally measured from the onset of the pacing stimulus or activation 111 in one atrium to the end of atrial depolarization in another atrium. The duration of the P-wave in one lead of the ECG is that part of the total atrial activation that can be detected on the body surface from one vantage point. Another ECG lead, however, may record a P-wave that may be shorter or longer, may start earlier or later, and/or may end earlier or later. Thus, the total atrial activation time is a more complete measure of the duration of atrial depolarization.

The total atrial activation time is the interval from the first onset of atrial tissue depolarization typically in the right atrium until the final depolarization of atrial tissue typically in the left atrium near the artrioventricular grove. The total duration of atrial depolarization is typically measured within the heart starting with the first recognized deviation from the base line (or pacer spike) on an electrogram recorded from an electrode in the first atrium until the final return to base line of the electogram recorded from a second electrode in the second atrium. The total atrial activation time is longer than the P-wave recorded from any single lead on the ECG.

The atrial conduction time (also referred to as the “inter-atrial conduction time”, “inter-atrial activation time”, or intra-atrial conduction time) is the interval between the onset or peak of atrial activity in the electrogram from the first electrode in a first atrium to the onset or peak of electrical activity atrial activity on the electrogram from the second electrode in the second atrium.

Following the P-wave is an isoelectric (zero voltage) period called the atrio-ventricular delay 102 (herein referred to as “AV delay” or “A-V delay”). The AV delay 102 is the time period between atrial electrical activity 100 and electrical activity in the ventricles 103. In pacemakers that provide stimulation pulses to one or more ventricles, the pacemaker provides stimulation only after the AV delay time 102 has expired. The period of time from the onset of the P-wave to the beginning of the QRS complex 103 is termed the P-R interval 101, which normally ranges from about 120 ms to about 200 ms in duration. The P-R interval 101 represents the time between the onset of atrial depolarization and the onset of ventricular depolarization. The QRS complex 103 represents ventricular depolarization and is normally from about 50 ms to about 100 ms in duration.

FIG. 1A is a diagram of two recordings 120A and 130A from electrodes attached to the atria in a patient in sinus rhythm with normal inter-atrial conduction time and normal total atrial activation time.

The first line 120A represents the electrogram recorded by a first electrode directly from the heart and, in one embodiment, measured from the right atrium. The peak deflection in the electrogram represents the electrical pulse created by depolarization of the atrial tissue in close proximity to the electrode. The onset of deflection 121A, end of deflection 122A, peak positive 123A, and total duration 124A of the deflection in the electrogram are shown accordingly.

The second line 130A represents the electrogram recorded by a second electrode directly from the heart and, in one embodiment, measured from the left atrium. The peak deflection in the electrogram represents the electrical pulse created by depolarization of the atrial tissue in close proximity to the electrode. The onset of deflection 131A, end of deflection 132A, peak positive 133A, and total duration 134A of the deflection in the electrogram are shown. The time interval 141A shows the interval between peak positive 133A recorded from the second electrode and the end of atrial depolarization 132A.

The total atrial activation time 113A is measured from 121A to 132A.

FIG. 1B is a diagram of two intra-cardiac recordings 120B and 130B from electrodes attached to the atria in a patient in sinus rhythm with prolonged inter-atrial conduction time and prolonged total atrial activation time.

The first line 120B represents the electrogram recorded by the first electrode directly from the heart and, in one embodiment, measured from the right atrium. The deflection in the electrogram represents the electrical pulse created by depolarization of the atrial tissue in close proximity to the electrode. The onset of deflection 121B, end of deflection 122B, peak positive 123B, and total duration 124B of the deflection in the electrogram are defined.

The second line 130B represents the electrogram recorded by the second electrode directly from the heart and, in one embodiment, measured from the left atrium. The deflection in the electrogram represents the electrical pulse created by depolarization of the atrial tissue in close proximity to the electrode. The onset of deflection 131B, end of deflection 132B, peak positive 133B, and total duration 134B of the deflection in the electrogram are defined. The time 141B is the interval between peak positive 133B recorded from the second electrode and the end of atrial depolarization 132B.

The total atrial activation time 113B is measured from 121B to 132B.

FIG. 1C is a diagram of two intra-cardiac recordings 120C and 130C from electrodes attached to the atria in a patient whose atrium is being paced using a right atrial electrode 120C.

The first line 120C represents the electrogram recorded by the first electrode directly from the heart typically the right atrium. The deflection in the electrogram 121C represents the electrical pulse created by the pacing pulse.

The second line 130C represents the electrogram recorded by the second electrode directly from the heart and, in one embodiment, measured from the left atrium. The deflection in the electrogram represents the electrical pulse created by depolarization of the atrial tissue in close proximity to the electrode. The onset of deflection 131C, end of deflection 132C, peak positive 133C, and total duration 134C of the deflection in the electrogram are defined. The time 141C is the interval between peak positive 133C recorded from the second electrode and the end of atrial depolarization 132C.

The total atrial activation time 113C is recorded from the pacer pulse 121C to the end of left atrial activation 132C.

It has been realized through the present invention that when the first electrode or lead is recording electrograms in the right atrium near the sinus node and the second electrode or lead is recording electrograms from the base of the left atrium in a mid coronary sinus position the following intervals are nearly identical: 1) the total sinus atrial activation time 113A and 113B measured by hand at the time of implant from electrograms 120A and 130A or 120B and 130B; 2) the maximum P-wave duration measured from 12 vertically oriented surface leads; and 3) the echocardiography based atrio-ventricular delay determined during sinus rhythm. In addition, it has been realized that changes in the total atrial activation time in sinus rhythm 113A or 113B or pacing 113C will be reflected in the inter-atrial conduction times 140A, 140B, or 140C measured as the interval between the peak deflections 123A and 133A, 123B and 133B, or pacer spike and 133C in the electrograms recorded from the two atria. That is, when the total inter-atrial activation time is short 113A (as in FIG. 1A), the interval between the peak deflections 123A and 133A on the second electrode is likewise short. When the total atrial activation time 113B is longer (as in FIG. 1B), the interval between the peak deflections 123B and 133B is likewise longer. Therefore, changes in the total atrial activation time and, thus, changes in the AV delay, can be derived from the inter-atrial activation time, measured electronically by the microcontroller of the current invention, to maintain the appropriate AV delay for optimal filling for that individual patient. One skilled in the art would recognize that LA pacing may be directed by the microcontroller because of a prolonged inter-atrial conduction time to insure optimal pacing in cardiac resynchronization therapy.

Similarly, it has been realized through the present invention that when the first lead is pacing the right atrium near the sinus node and the second lead is recording electrograms from the base of the left atrium in a mid coronary sinus position the following intervals are nearly identical: 1) the paced total atrial activation time 113C measured by hand at the time of implant from electrograms 120C and 130C; 2) the maximum paced P wave duration measured from 12 vertically oriented surface leads; 3) the echocardiography based atrio-ventricular delay determined during atrial pacing. In addition, it has been realized that changes in the total atrial activation time during pacing 113C can be derived from changes in the paced inter-atrial conduction time 140C measured electronically as the interval between the pacing pulse 121C and the peak of the deflection recorded from the left atrium 133C. That is, when the paced inter-atrial activation time is reduced, the total atrial activation is also reduced. When the total atrial activation time is longer, the interval between the pacer spike and the peak deflection on the second electrode is likewise longer. Thus, during pacing, changes in the total atrial activation time (and thus the AV delay) can be derived from changes in the inter-atrial activation time measured electronically by the microcontroller of the current invention and used to set the appropriate AV delay for that patient.

Indeed, the inter-atrial conduction delay is variable according to each individual and, for each person, changes in biological or environmental conditions such as age, health, general condition, sleep-wake cycle, illness, medication, diet, stress, and/or the effort (activity level) of the patient. As a result, the inter-atrial activation time measured electronically by the microcontroller from the deflection or pacing pulse in the right atrium (near the sinus node) to the deflection in the electrogram from the electrode recording cardiac activity in the left atrium (from the mid coronary sinus) eliminates the need for empirical or echocardiography based optimization routines while providing optimized atrio-ventricular delays for the patients' present condition.

In one embodiment of the current invention, the total atrial conduction time, which has the same duration as the optimal atrio-ventricular delay for CRT, is used to set the atrio-ventricular delay. The total atrial activation may be determined manually at the time of implant or from an algorithm based on the relationship between the inter-atrial activation time and the total atrial activation time. As previously mentioned, the inter-atrial conduction time is measured by the onset or peak of activation between an electrical pulses in a first atrium and a second atrium. Changes in the total atrial activation time and, thus, changes in the AV delay, can be derived from changes in the inter-atrial activation time, measured electronically by the microcontroller of the current invention, to maintain the appropriate AV delay for that individual patient.

In another embodiment of the current invention, the total atrial activation time, and thus the optimal atrio-ventricular delay for CRT can be derived through signal processing of the inter-atrial electograms recorded from electrodes in a first atrium and a second atrium. According to this method, the onset of all electrical activity is determined by signal processing from the electrogram recorded in a first atrial chamber and the end of all electrical activity is determined by signal processing from the electrogram in a second atrial chamber.

In yet another embodiment of the current invention, the microcontroller of the current invention would monitor the electrogram sensed by the left atrial electrode to determine the end of left atrial activation through signal processing. The end of left atrial activation may be the peak activation, local activation, or a more remote activation. One skilled in the art would recognize that the total atrial activation is able to be derived from the measured end of left atrial activation and the measured inter-atrial conduction time. As previously mentioned, the inter-atrial conduction time may be measured by observing the onset or peak of activation between electrical pulses in a first atrial chamber and a second atrial chamber.

Indeed, one skilled in the art would recognize that there are a variety of methods in which the atrioventricular delay may be derived simply by measuring parameters including the inter-atrial activation time, the total inter-atrial conduction time, the intervals 141A, 141B, or 141C, the peak activations in each atrium arbitrary or defined sub-periods within these activation periods, or combinations thereof. Further, once such parameters are measured, one skilled in the art would recognize that from the various relationships between the parameters, algorithms or signal processing may be applied such that the optimal atrio-ventricular delay may be derived. Moreover, one skilled in the art would recognize that any method or device utilizing the changes in inter-atrial conduction time, in conjunction with the other parameters enumerated above, can be used to derive the total atrial activation time or other delay periods related to the total atrial activation time, and, thus, the optimal atrio-ventricular delay.

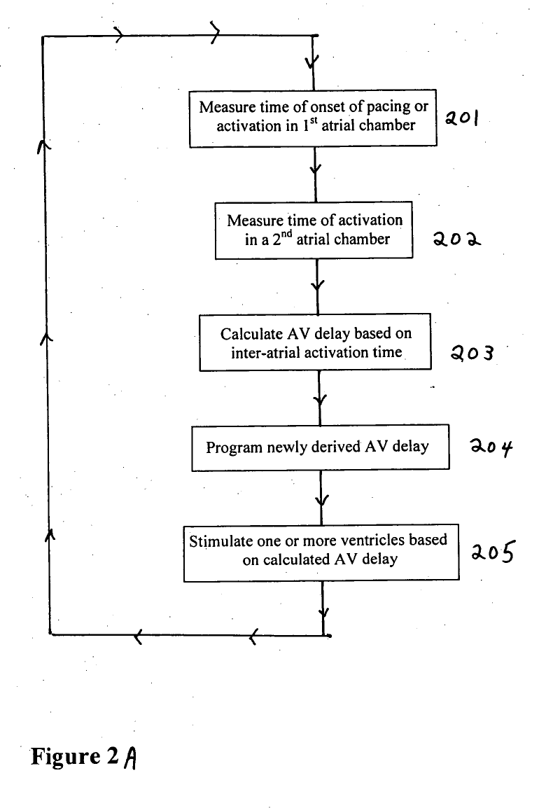

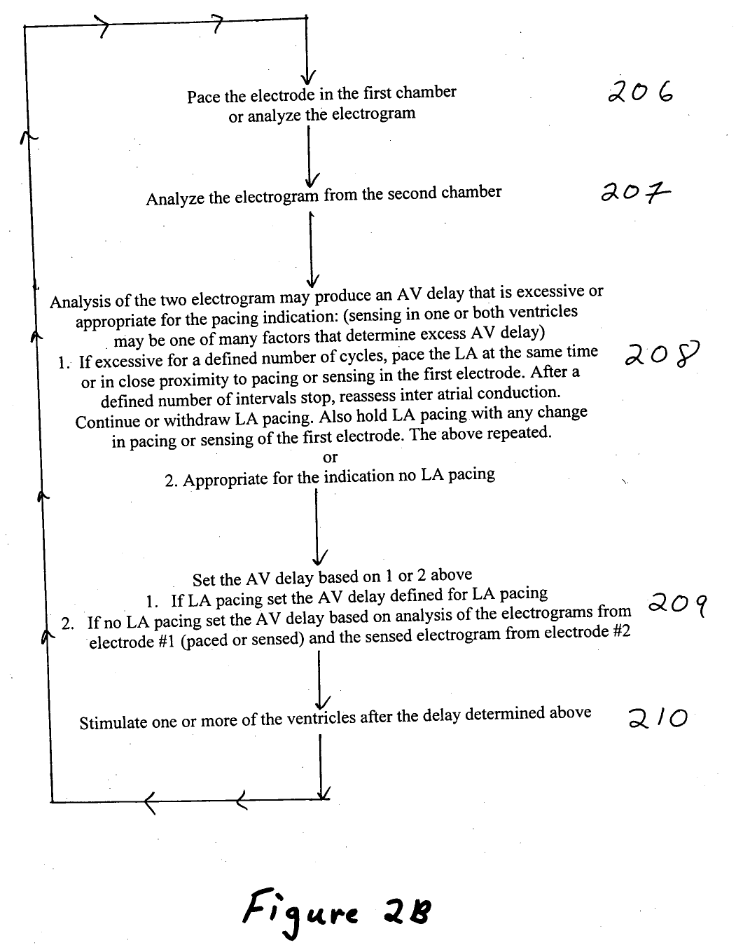

FIG. 2A is a flowchart illustrating the basic functioning of a cardiac device employing the improvement of the current invention. At step 201, the cardiac device either measures the time of onset of atrial activation in a first atrial chamber or paces if no atrial activation is detected after a preset interval. At step 202, the cardiac device measures the time of atrial activation in a second atrial chamber. The cardiac device then calculates 203 the inter-atrial conduction time based on the aforementioned time measurements. Changes in the interatrial conduction time can be used to adjust the AV delay accordingly. For the individual patient, the interval (141A, 141B, or 141C) added to the AV delay is proportional to the inter-atrial conduction time (140A, 140B, or 140C) measured by the microcontroller. Finally, the cardiac device is programmed 204 with a new atrio-ventricular delay time based on the inter-atrial activation time. Stimulation 205 is delivered to one or more ventricles based on the programmed atrio-ventricular delay time. If the microcontroller senses activation of the ventricle from any electrode attached to either ventricle before a pre-specified interval, stimulation will be withheld. The process repeats (steps 201-205) allowing for a dynamically programmed optimal AV filling based on the AV delay and pacing (or lack thereof) of the LA that is patient specific and responsive to changes in patient activity level and stresses. In a preferred embodiment, the process is monitored continuously. Continuously programming the LA pacing on or off and the atrio-ventricular delay based on analysis of electograms recorded from electrodes in the RA and LA ensures that the AV delay is not set too short (so as to prevent high LV pressures) and ensures that the AV delay is not set too long (for example, so as to provide for optimal LV filling).

FIG. 2B is a flowchart illustrating an alternate method of the basic functioning of a cardiac device employing the improvement of the current invention. At step 206, the electrode in the first chamber is paced or the electrogram is analyzed. At step 207, the electrogram from the second chamber is analyzed. At step 208, the analysis of the two electrograms may show either an excessive or an appropriate AV delay for the pacing indication. If the AV delay is excessive for a defined number of cycles, the LA should be paced at the same time or in close proximity to the pacing or the sensing in the first electrode. After a defined number of intervals, pacing could be stopped and the inter-atrial conduction would be reassessed. At this point, LA pacing could either be continued or withdrawn. Moreover, the LA pacing should be held if there is any change in the pacing or sensing of the first electrode. However, if the AV delay is appropriate for the pacing indication, then no LA pacing is necessary. At step 209, the AV delay is set based on step 208. If the cardiac device is LA pacing, the AV delay should be set as defined for LA pacing. However, if the cardiac device is not LA pacing, then the AV delay should be set based on the analysis of the electogram from a first paced or sensed first electrode and the electrogram from a second sensed electrode. Finally, at step 210, one or more ventricles are stimulated based on the aforementioned delay.

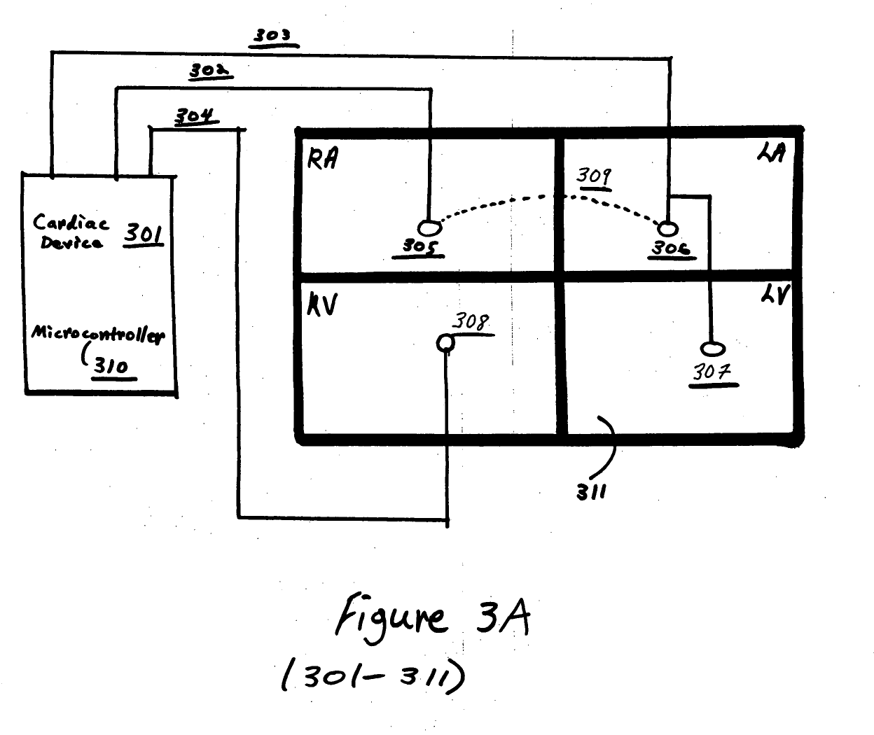

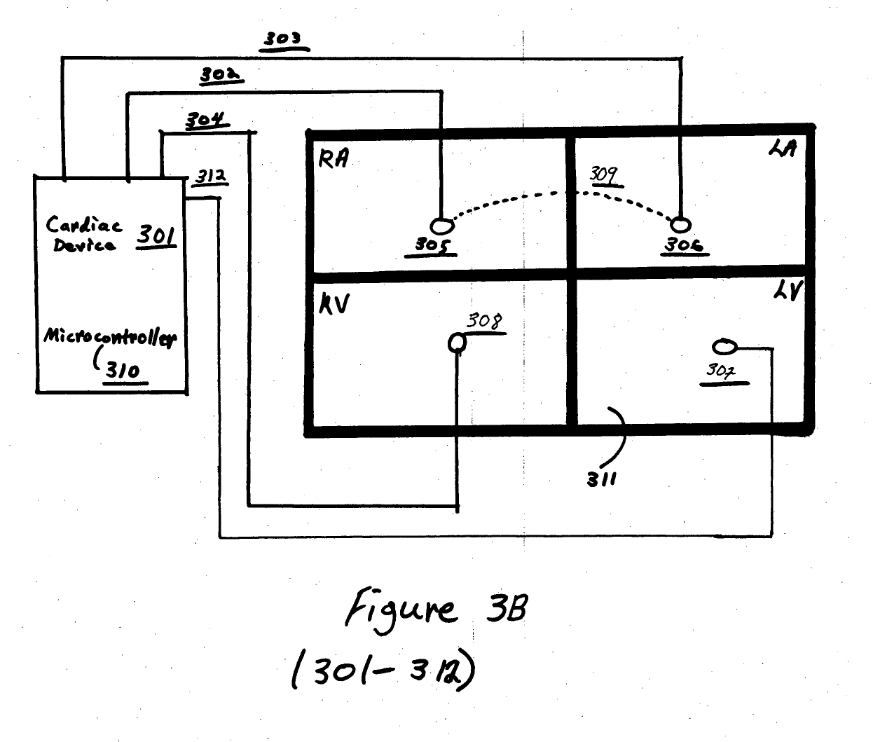

FIG. 3A illustrates a diagrammatic representation of a cardiac device 301 in electrical communication with a patient's heart 311 by way of three leads 302, 303, and 304. While this cardiac device is depicted with three leads for illustration purposes, one of skill in the art could appropriately adapt any two, three, or four lead cardiac device to provide appropriate cardiac therapy in furtherance of utilizing the methods and apparatus of the present invention. The leads utilized as part of the current invention may be single or compound leads. A compound lead contains one or more individual leads that connect separately to the individual independent inputs or outputs on the cardiac device. Thus, in one embodiment, a compound lead may be utilized having one or more individual leads with each individual lead having one or more electrodes.

Lead 302 comprises at least one electrode 305 for sensing cardiac events in a first atrium. In one embodiment, lead 302 terminates in a right atrium with a single electrode. As depicted in FIG. 3A, lead 303 is a compound lead. Lead 303 comprises at least one electrode 306 for sensing cardiac events in a second atrium and/or to provide stimulation to a second atrium. Lead 303 may be a compound electrode also contains a second electrode 307 for sensing cardiac events in a ventricle or to provide stimulation to a ventricle. In one embodiment, lead 303 is a compound electrode that has two electrodes, one of which terminates in a left atrium and the second of which terminates in a left ventricle.

FIG. 3B illustrates a diagrammatic representation of a cardiac device 310 in electrical communication with a patient's heart 311 by way of four leads 302, 303, 304, and 312. While this cardiac device is depicted with four leads for illustration purposes, one of skill in the art could appropriately adapt any two, three, or four lead cardiac device to provide appropriate cardiac therapy. As depicted in FIG. 3B, lead 303 comprises at least one electrode 306 for sensing cardiac events in a second atrium and/or to provide stimulation to a second atrium. Moreover, lead 312 comprises at least one electrode 307 for sensing cardiac events in a ventricle or to provide stimulation to at least one ventricle.

To provide dual chamber stimulation therapy, the cardiac device 301 contains lead 304. Lead 304 comprises at least one electrode 308 which senses cardiac events and/or provides stimulation to one or more heart chambers. In one embodiment, lead 304 comprises at least one electrode 308 for sensing cardiac events in a ventricle or for providing stimulation therapy to at least one ventricle. In another embodiment of the invention, lead 304 has one electrode which provides stimulation therapy to a right ventricle.

The cardiac device 301 contains a microcontroller 310 and related circuitry which measures the total inter-atrial conduction time delay 309 (as shown by a dashed lined) between a first atrial chamber and a second atrial chamber. Analysis of the electograms from the electrodes in the RA and LA is used to determine if LA pacing is required and the optimal atrio-ventricular delay. In one embodiment of the invention, the cardiac device 301 measures the conduction time delay 309 between electrode 305 and electrode 306. However, it can be appreciated by one skilled in the art that the inter-atrial conduction delay may be measured between by any single compound electrode in any chamber or by any plurality of electrodes in any chamber. Stimulation therapy may be provided to one or more chambers of the heart via electrodes 307 and/or 308 based on the need for LA pacing and the derived atrio-ventricular delay.

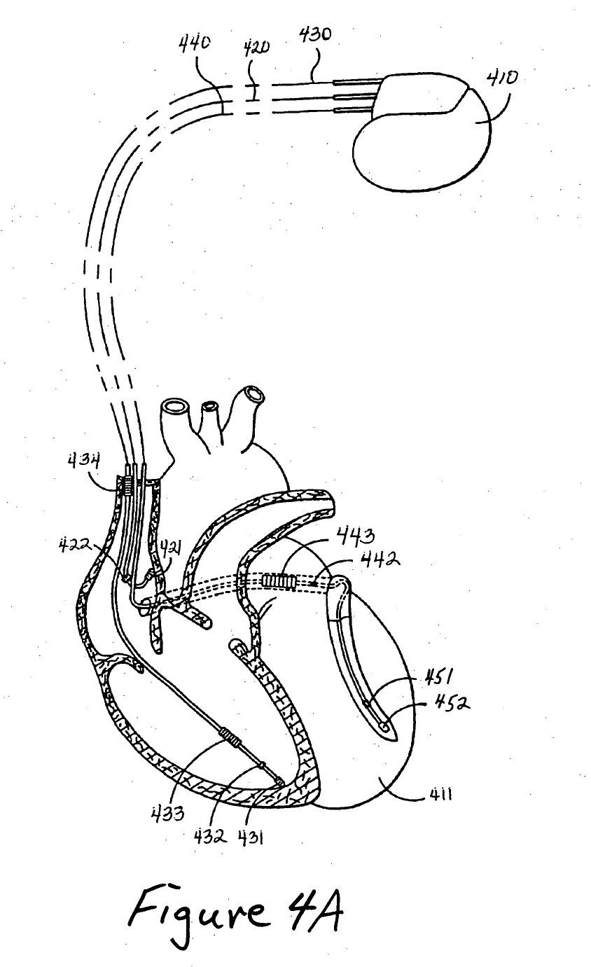

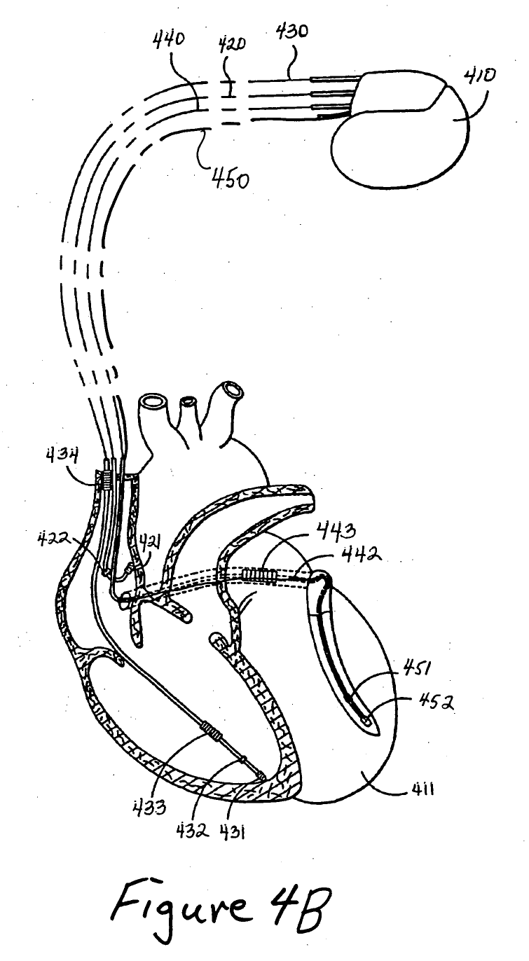

FIG. 4A illustrates a more detailed view of a cardiac device 410 in electrical communication with a patient's heart 411 by way of three leads 420, 430 and 440 suitable for delivering multi-chamber stimulation and shock therapy. While a particular three-lead multi-chamber cardiac device is shown for illustration purposes, one of skill in the art could readily duplicate, eliminate or disable the appropriate circuitry in any desired combination to provide a device capable of treating the appropriate chamber(s) of the heart with cardioversion, defibrillation and/or pacing stimulation. One skilled in the art will recognize that the present invention is not limited solely to three lead cardiac devices, and that two or four lead pacemakers make be similarly modified to provide the desired therapeutic capability. The leads exiting the pacemaker may be compound leads containing two or more individual leads that connect separately to individual independent input/outputs on the device 410. That is, a single compound lead may contain any number of independent leads each having one or more electrodes. As shown in FIG. 4A, lead 440 is a compound lead having two individual leads each terminating in one or more electrodes.

FIG. 4B illustrates a more detailed view of a cardiac device 410 in electrical communication with a patient's heart 411 by way of four leads 420, 430, 440, and 450 suitable for delivering multi-chamber stimulation and shock therapy. While a particular four-lead multi-chamber cardiac device is shown, this is for illustration purposes only and one of skill in the art could readily duplicate, eliminate or disable the appropriate circuitry in any desired combination to provide a device capable of treating the appropriate chamber(s) of the heart with cardioversion, defibrillation and/or pacing stimulation. One skilled in the art will recognize that the present invention is not limited solely to four lead cardiac devices, and that any two or three lead pacemakers with three or more independent input/outputs channels may be similarly modified to provide the desired therapeutic capability. As such, any of the leads depicted in FIG. 4B may be compound leads.

To sense atrial cardiac signals and to provide right atrial chamber stimulation therapy, the stimulation device 410 is coupled to an implantable right atrial lead 420 having at least an atrial tip electrode 421, which typically is implanted in the patient's right atrial appendage. Alternative locations for the one or more right atrial electrodes include Bachman's Bundle or the Triangle of Koch. The right atrial lead 420 may also have a right atrial ring electrode 422 to allow bipolar stimulation or sensing in combination with the right atrial tip electrode 421.

To sense left atrial and ventricular cardiac signals and to provide left-chamber stimulation therapy, the stimulation device 410 is coupled to a “coronary sinus” (“CS”) lead 440 designed for placement in the “coronary sinus region” via the coronary sinus OS so as to place one or more electrodes adjacent to the left atrium. For example, a pacing lead 440 placed in the coronary sinus may be a compound lead having one or more independent leads placed in proximity to the left atrium and/or the left ventricle through a coronary vein to pace, sense or stimulate the left atrium and/or the left ventricle (as in FIG. 4A). In an alternate example, a second coronary sinus lead 450 may be placed in the coronary sinus in conjunction with lead 440, such that one or more electrodes are placed in proximity to the left ventricle and the left atrium (as in FIG. 4B). As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. While the mid CS is the preferred location for the left atrial lead, alternative locations for the left atrial electrode include the proximal or mid main CS and the Triangle of Koch. However, changes in the inter-atrial conduction recorded from these alternative locations may not allow as accurate a calculation of the AV delay.

In one embodiment, when left ventricular stimulation is not required, a single individual lead 440 having at least one electrode 442 may be placed in the mid CS for pacing and sensing the left atrium. The at least one electrode 442 may be a ring electrode or a tip electrode. However, a ring electrode for bipolar pacing is desirable to insure that ventricular fibrillation detection is not inhibited.

In another embodiment, when left ventricular stimulation is required, a second independent lead is needed, which may be part of a compound lead. Thus, in one embodiment lead 440 is a compound lead providing individual leads to each of the left atrium and the left ventricle (FIG. 4A). In another embodiment, lead 440 is an individual lead providing one or more electrodes to the left atrium and lead 450 is an individual lead providing one or more electrodes to a left ventricle (FIG. 4B). If the one or more electrodes for left ventricular pacing are contained on a lead separate from the left atrial lead it may have at least a tip electrode 452, although a ring electrode 451 for pacing and sensing may be added. Placing two separate leads into the coronary sinus, such as in FIG. 4B, may be time consuming and difficult. Thus, in one embodiment, it is preferable to have a single compound lead pace and sense both the left atrium and the left ventricle, such as in FIG. 4A.

Accordingly, compound coronary sinus leads may be designed to receive independent atrial and ventricular cardiac signals and to deliver: left ventricular pacing therapy using at least a left ventricular tip electrode 452, left atrial sensing or pacing therapy using at least a left atrial ring electrode 442, and/or shocking therapy using at least a left atrial coil electrode 443. In an alternative embodiment, the compound coronary sinus lead for left atrial pacing and sensing and left ventricular pacing and sensing may also include a left ventricular ring electrode 451 and a second left atrial ring electrode 442.

The cardiac device 410 is also shown in electrical communication with the patient's heart 411 by way of an implantable right ventricular lead 430 having, in this particular embodiment, a right ventricular tip electrode 431, a right ventricular ring electrode 432, a right ventricular (RV) coil electrode 433, and/or an SVC coil electrode 434. Typically, the right ventricular lead 430 is transvenously inserted into the heart 411 so as to place the right ventricular tip electrode 431 in the right ventricular apex so that the RV coil electrode 433 will be positioned in the right ventricle and the SVC coil electrode 434 will be positioned in the superior vena cava. Accordingly, the right ventricular lead 430 is capable of receiving cardiac signals, and delivering stimulation in the form of pacing and shock therapy to the right ventricle.

As with the left ventricle and left atrium, a compound lead (not shown) may be designed to receive independent right atrial and right ventricular cardiac signals and to deliver: right ventricular pacing therapy using a right ventricular tip electrode 431, right atrial sensing or pacing therapy using a right atrial ring electrode 422, and/or shocking therapy using a right atrial coil electrode 434.

At least one independent lead, regardless of whether it is part of a compound lead, must be attached to one of the ventricles. Depending on the patient's cardiac condition it may be preferable to pace both the right ventricle and left ventricle. When only a single electrode is placed in the ventricles, pacing the right ventricle is technically less demanding than pacing the left ventricle.

The cardiac device 410 includes a housing which may be programmably selected to act as the return electrode for all “unipolar” modes. The cardiac device housing may further be used as a return input/output alone or in combination with one or more of the coil electrodes for shocking purposes.

The cardiac device 410 further includes a connector having a plurality of terminals which may include terminals for a LV tip electrode, a LV ring electrode, a LA ring electrode, LA coil electrode, a RA tip electrode, a RA ring electrode, a RV ring electrode, a RV tip electrode, a RV coil electrode, a CS coil electrode, and/or a SVC coil electrode. Again, while a particular multi-chamber cardiac device is described, this is for illustration purposes only, and one of skill in the art could readily duplicate, eliminate or disable the appropriate circuitry in any desired combination to provide a device capable of treating the appropriate chamber(s) of the heart with cardioversion, defibrillation and/or pacing stimulation.

To achieve RA sensing and pacing, the connector includes at least a right atrial tip electrode adapted for connection to the atrial tip electrode 421. The connector may also include a right atrial ring terminal for connection to the atrial ring electrode 422, and a left ventricular ring terminal for connection to the left ventricular ring electrode 444.

To achieve left chamber sensing, pacing and/or shocking, the connector includes at least a left ventricular tip terminal, a left atrial ring terminal, and a left atrial shocking terminal, which are adapted for connection to the left ventricular tip electrode 441, the left atrial ring electrode 442, and the left atrial coil electrode 443, respectively.

To support right chamber sensing, pacing and/or shocking, the connector further includes a right ventricular tip terminal, a right ventricular ring terminal, and right ventricular shocking terminal, and an SVC shocking terminal, which are adapted for connection to the right ventricular tip electrode 431, right ventricular ring electrode 432, the RV coil electrode 433, and the SVC coil electrode 434, respectively.

At the core of the cardiac device 410 is a programmable microcontroller that controls the various modes of stimulation therapy. The microcontroller typically includes a microprocessor, or equivalent control circuitry, designed specifically for controlling the delivery of stimulation therapy, and may further include RAM or ROM memory, logic and timing circuitry, state machine circuitry, and I/O circuitry. Typically, the microcontroller includes the ability to process or monitor input signals (data) as controlled by a program code stored in a designated block of memory. For example, the microcontroller may sense electrical cardiac activity and make measurements according to such sensed activity. Based on any measured or derived data processed by the microcontroller, the microcontroller may control pacing, stimulation, or shocking therapy to one or more chambers of the heart. Any suitable microcontroller may be used that carries out the functions described herein.

The microcontroller further includes timing control circuitry which is used to control the timing of such electrical or stimulation pulses (e.g. pacing rate, atrio-ventricular (AV) delay, inter-atrial conduction time (A-A) delay, or ventricular interconduction (V-V) delay, etc.), as well as to keep track of the timing of refractory periods, PVARP intervals, noise detection windows, evoked response windows, alert intervals, marker channel timing, etc.

The cardiac device 410 further includes one or more atrial and ventricular sensing circuits, known in the art, for detecting the presence of cardiac activity in each of the four chambers of the heart. The cardiac device may also contain one or more physiologic sensors, often referred to as “rate-responsive” sensors. The physiological sensor may be used to detect changes in cardiac output, changes in the physiological condition of the heart, or diurnal changes in activity. The microcontroller responds to sensed signals by adjusting the various pacing parameters, including AV delay.

The cardiac device 410 further includes atrial and ventricular pulse generators so as to provide pacing stimulation pulses for delivery by the right atrial lead, the right ventricle lead and/or the coronary sinus lead. The cardiac device further includes a switch which includes a plurality of switches for connecting the desired electrodes to the appropriate I/O circuits, thereby providing complete electrode programmability.

In one embodiment, a cardiac device is provided comprising a first electrode for sensing an electrical pulse in an atrium, a second electrode for sensing an electrical pulse in an atrium, a microcontroller coupled to the first electrode and the second electrode for determining time between the first and second pulses, and an electrode for stimulating a chamber of a heart wherein the stimulation is based on the first and second pulses. The sensed interval between the first sensed or paced pulse and the second sensed pulse is the inter-atrial conduction time when the electrodes are in different atria and intra atrial conduction time if the two electrodes are in the same atrial chamber.

In another embodiment, a cardiac device is provided comprising a microcontroller, a first lead in communication with the microcontroller having at least a first electrode for pacing or sensing cardiac events in a first atrium (e.g.: electrode 421 or 422), a second lead in communication with the microcontroller having at least a second electrode for pacing or sensing cardiac events in a second atrium (e.g.: electrode 442 or 443), and at least one stimulating electrode for providing stimulation pulses to one or more heart chambers, whereby the microcontroller measures a delay between cardiac events sensed by the first and second electrodes, and whereby the microcontroller directs the stimulation pulse based on the delay. The sensed interval between the first sensed or paced pulse and the second sensed pulse is the inter-atrial conduction time when the leads are in different atria and intra atrial conduction time if the two electrodes are in the same atrial chamber.

In a first embodiment, the microcontroller coupled to the one or more electrodes may calculate the inter-atrial conduction time between an electrode in a first atrium and an electrode in a second atrium. In one embodiment, the first and second atria may be the same atrium. In a preferred embodiment of the invention, the first and second atria may be different atrial chambers. When the first and second atria are different atrial chambers, it is preferred that the first atrial chamber is the right atrium and the second atrial chamber is the left atrium. For two electrode systems, it can be appreciated by one skilled in the art that the inter-atrial conduction time may be measured between any two electrodes regardless of whether the electrodes are of the same type and regardless of the location of the electrodes.

After the microcontroller calculates the inter-atrial conduction time as measured between the first and the second atrial chambers, the microcontroller may store the inter-atrial conduction time in memory. From the measured inter-atrial conduction time, the atrio-ventricular delay may be derived by the microcontroller. The AV delay may then be stored within the cardiac device.

The microcontroller may then direct the delivery of stimulation pulses to one or more chambers of the heart based on the derived atrio-ventricular delay. For example, one or more stimulation pulses may be delivered via electrodes 431, 432, 441, and/or 444 to one or more heart chambers based on the derived AV delay. In one embodiment of the invention, stimulation pulses may be delivered only to the left ventricle or only to the right ventricle. In another embodiment of the invention, stimulation pulses may be delivered to the left ventricle and the right ventricle. The microcontroller may also direct shocking therapy to one or more chambers of the heart based on the derived AV delay.

In yet another embodiment, a cardiac device is provided comprising a microcontroller, a first lead connected to the microcontroller having at least a first electrode for sensing cardiac events or pulsing terminating in a right atrium, a second lead connected to the microcontroller having at least a second electrode for sensing cardiac events or pulsing terminating in a left atrium, and at least one electrode for sensing cardiac events or pulsing terminating in a left ventricle, whereby the microcontroller measures a delay between paced or sensed cardiac events sensed by the first electrode and the second electrode, and whereby the microcontroller directs a stimulation based on the measured delay. The stimulation pulse to the ventricles may be to one or both ventricles. When stimulation of two ventricles occurs, they may be paced either simultaneously or with a delay. Each ventricle may be stimulated at one or multiple locations. The microprocessor may cancel the stimulation pulse to one or both of the ventricle(s) if one or more of the electrodes in the ventricles sense a cardiac event occurring in the one or both ventricles before the end of the AV delay determined by the inter-atrial measurement. The measured delay between cardiac events sensed by the first and second electrodes is the inter-atrial conduction time or the time from the electronically detected onset of activation in a first atrium to the electronically detected onset of activation in a second atrium.

It can be appreciated by one skilled in the art that the inter-atrial conduction time is variable according to each individual and changes with biological and environmental conditions such as effort and loading conditions. Indeed, the inter-atrial delay may vary as the body responds to different heart rates, loads, chemicals, or stresses. Thus, as the inter-atrial conduction time changes, there is a need to dynamically change the atrio-ventricular delay. Thus, in one embodiment of the current invention, the inter-atrial conduction time is continuously monitored and new atrio-ventricular delays are derived from the changing inter-atrial conduction times. The dynamically derived atrio-ventricular delay is then programmed into the cardiac device. Pacing, stimulating, or shocking therapy may be delivered to one or more heart chambers according to the dynamically derived atrio-ventricular delay.

In another embodiment, a cardiac device is provided comprising a microcontroller, a compound lead connected to the microcontroller, one or more individual leads connected to the compound lead, one or more electrodes connected to each individual lead for pacing or sensing cardiac events, and at least one stimulating electrode for providing stimulation pulses to one or more heart chambers, whereby the microcontroller measures a delay between paced or sensed cardiac events between the one or more electrodes, and whereby the microcontroller directs the stimulation pulse based on the delay.

In a further embodiment of the current invention, the electrodes may be wireless. Accordingly, such electrodes may be positioned in one or more heart chambers to sense, pace, or shock one or more chambers of the heart without being directly connected to the cardiac device via a lead. Measurement of the inter-atrial conduction time and subsequent derivation of the AV delay would proceed in the same manner as a cardiac device having one or more leads connected to the various electrodes. Wireless electrodes may be of the same types found in cardiac devices having leads including, but not limited to, tip electrodes, ring electrodes, and coils.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cardiac device comprising:
a first sensing electrode for sensing an electrical pulse in an atrium,
a second sensing electrode for sensing an electrical pulse in an atrium,
a microcontroller in communication with said first electrode and said second electrode for determining a delay between said first and second pulses, and
a stimulating electrode for providing a stimulation to a chamber of a heart,
wherein the timing for said stimulation delivered by said stimulating electrode is based on said delay between said first and second pulses.

2. The cardiac device of claim 1, wherein said first electrode and said second electrode are in the same atrium.

3. The cardiac device of claim 1, wherein said first electrode is in a right atrium and said second electrode is in a left atrium.

4. The cardiac device of claim 3, wherein said electrode for stimulating is a third electrode positioned in a ventricle.

5. The cardiac device of claim 1, wherein said delay is successively measured over intervals of time.

6. The cardiac device of claim 1, further comprising at least one coil for providing shocking therapy to one or more heart chambers.

7. A cardiac device comprising:
a microcontroller,
a first lead in communication with said microcontroller having at least a first electrode for sensing cardiac events in a first atrium,
a second lead in communication with said microcontroller having at least a second electrode for sensing cardiac events in a second atrium, and
at least one stimulating electrode for providing stimulation pulses to one or more heart chambers,
whereby said microcontroller measures a delay between cardiac events sensed by said first and second electrodes, and
whereby said microcontroller directs said stimulation pulse based on said delay.

8. The cardiac device of claim 7, wherein said first atrium and said second atrium are the same atria.

9. The cardiac device of claim 7, wherein said first and second leads are connected to a compound lead and said compound lead is connected to said microcontroller.

10. The cardiac device of claim 7, wherein said first atrium is the right atrium and said second atrium is the left atrium.

11. The cardiac device of claim 7, wherein said stimulating electrode is a third electrode in communication with said second lead.

12. The cardiac device of claim 7, further comprising one or more coils for providing shocking therapy to one or more chambers of the heart.

13. The cardiac device of claim 7, further comprising a third lead in communication with said microcontroller, having at least one electrode for sensing cardiac events, for stimulating one or more heart chambers, or for providing shocking therapy to one or more heart chambers.

14. A method of optimizing the pacing methodology of a cardiac device comprising:
measuring an onset of atrial activation time in a first atrium,
measuring the onset of atrial activation time in a second atrium,
calculating the inter-atrial conduction time between said first and second atria based on said measured activation times,
deriving said atrio-ventricular delay from said calculated inter-atrial conduction time,
programming said atrio-ventricular delay into said cardiac device.

15. The method of claim 14, wherein said first atrium and said second atrium are the same.

16. The method of claim 14, wherein said first atrium and said second atrium are different.

17. The method of claim 14, wherein said first atrium is a right atrium and said second atrium is a left atrium.

18. The method of claim 14, wherein said atrio-ventricular delay is programmed at the time of surgery.

19. The method of claim 14, wherein said atrio-ventricular delay is programmed dynamically.

20. The method of claim 14, wherein said inter-atrial conduction time is measured by at least one electrode.
